SEQUENCE LISTING (36 sequences)

SEQ ID NO: 1 - DNA, 31 bp, synthetic construct
  source 1..31: mol_type = other DNA
tttacactttatgcttccggctcgtatgttg

SEQ ID NO: 2 - DNA, 1755 bp, synthetic construct
  source 1..1755: mol_type = other DNA
atgaacagtcagcaaaatcctgatttagctgttcatcccttggcaattcctatggaaggcttactaggagaaagtgctacaactcttgagaagaatgtaattgccacacaactctcagaggaagcccaagtaaagctagaggtaatccaaagtttactggaaccctgcgatcgcacaacttatgggcaaaagttgcgggaagcagcagagaaactaaatgtatcgttgcgaacggtacaaaggttggtgaaaaactgggaacaagatggcttagtcggactcactcaaacaagtagggctgataaaggaaaacaccgcattggtgagttttgggaaaacttcattaccaaaacctacaaggagggtaacaagggaagtaaacgtatgacccctaaacaagttgctctcagagtcgaggctaaagcccgtgaattaaaagactctaagccgcccaattacaaaaccgtgttacgggtattagcacccattttggaaaagcaacaaaaagccaagagtatccgcagtcctggttggagaggaactacgctttcggttaaaacccgtgaaggaaaagatttatcggttgattacagtaaccatgtttggcaatgtgaccatacccgcgtggatgtgttgctggtagatcaacatggtgaaattttaagtcgtccctggctaacaacagtaattgatacttactctcgttgcattatgggtatcaacttgggctttgatgcacccagttctggggtagtagcattagcgttacgccatgcaattctaccaaagcgttacggttccgagtacaaactgcattgtgagtggggaacctatggaaaaccagaacatttttatactgatggcggtaaagactttcgctctaaccacttgagtcagattggggcgcaattgggatttgtctgtcatttacgcgatcgcccttctgaaggtggagtagtagaacgtcccttcaaaacattaaatgaccaactattttcaacgcttcctgggtacaccggatctaatgtgcaggaacgcccagaagatgcagagaaggacgcaagacttactttgcgagaactagaacagttacttgtgcgttacatcgtagatcgttacaaccaaagtattgatgcgcggatgggcgaccaaacgcgctttgagcgttgggaagcaggattgcctacagtgccagtaccaataccagaacgagatttggatatttgtttaatgaagcagtcacggcgcactgtgcaaagaggtggttgtttgcagtttcagaatttaatgtatcggggggaatatttggcaggttatgccggagaaactgtcaacttaaggtttgaccccagagacattacaacaattttggtttatcgccaggaaaacaatcaggaagtatttctgactcgcgctcacgctcaaggtttggagacagagcaactggcattagatgaggctgaggcagcaagtcgcagactccgtaccgcagggaaaactatcagtaaccaatcattattgcaagaagttgttgaccgcgatgctcttgtcgctaccaagaaaagccgtaaggagcgtcaaaaattggaacagactgttttgcgatctgctgctgttgatgaaagtaatagagaatccttgccttctcaaatagttgaaccagatgaagtggaatctacagaaacggttcactctcaatacgaagacattgaggtgtgggactatgaacaacttcgtgaagaatatgggttttaa

SEQ ID NO: 3 - DNA, 831 bp, synthetic construct
  source 1..831: mol_type = other DNA
atgacagaagctcaggcgatcgccaagcagttgggtggggtaaaaccggatgatgagtggttacaagctgaaattgctcgtctcaagggtaagagcattgtgcctttacagcaggtaaaaactctccatgattggttagatggcaagcgcaaggcaagaaaatcttgccgagtagttggggaatcgagaactggcaagacagttgcttgtgatgcctacagatacaggcacaaacctcagcaggaagctggacgacctccaactgtgcctgtcgtttatattcgacctcaccaaaaatgtggccccaaggatttgtttaaaaagattactgagtacctcaagtatcgggtaacaaaagggactgtatctgattttcgagataggacgatagaagtactcaagggttgtggcgtagagatgctaattattgatgaagctgaccgtctcaagcctgaaacttttgctgatgtgcgagatattgccgaagatttaggaattgctgtggtactggtaggaacagaccgtttggatgcggtaattaagcgggatgagcaggttctcgaacgctttcgggcgcatcttcgctttggtaaattgtcgggagaggattttaagaacaccgtagaaatgtgggaacaaatggttttgaaactgccagtatcttctaatctaaagagcaaggagatgctacggattctcacgtcagcaactgaaggctacattggtcgccttgatgagattcttagggaagctgcaattcgttccttatcaagaggattgaagaagattgacaaggctgttttacaggaagtagctaaggagtacaaatga

SEQ ID NO: 4 - DNA, 504 bp, synthetic construct
  source 1..504: mol_type = other DNA
atgatagaagcaccagatgttaaaccttggctattcttgattaaaccctatgaaggggaaagcctgagccactttcttggcaggttcagacgtgccaaccatttatccgcaagtggattgggtactttggcaggaattggtgctatagtggcacgttgggaaagatttcattttaatcctcgccctagtcagcaagaattggaagcgatcgcatctgtagtagaagtggatgctcaaaggttagcccagatgttaccgcctgctggagtgggaatgcagcatgagccaattcgcttgtgtggggcttgttatgccgagtcgccttgtcaccgaattgaatggcagtacaagtcggtgtggaagtgcgatcgccatcaactcaagattttagcaaagtgtccaaactgtcaagcaccttttaaaatgcctgcgctgtgggaggatgggtgctgtcacagatgtaggatgccgtttgcagaaatggcaaagctacagaaggtttga

SEQ ID NO: 5 - DNA, 1920 bp, synthetic construct
  source 1..1920: mol_type = other DNA
atgagtcaaataactattcaagctcgacttatttcctttgaatcaaaccgccaacaactctggaagttgatggcagatttaaacacgccgttaattaacgaactgctttgccagttaggtcaacaccccgacttcgagaagtggcaacaaaagggtaaactcccgtctaccgttgtgagccagttatgtcaacctctcaaaactgaccctcgctttgcaggtcagcccagccgtttatatatgtcggcaattcatattgtggactacatctacaagtcctggctggctatacagaaacggcttcaacagcagctagatggaaagacgcgctggctagaaatgctcaatagcgatgctgaattagtagaacttagtggtgacactttagaggctattcgtgtcaaagctgctgaaattttggcaatagctatgccagcatctgagtcagatagcgcttcacctaaagggaaaaaaggtaaaaaggagaaaaaaccctcatcttctagccctaagcgtagtttatccaagacattatttgacgcttaccaagaaacggaagatatcaagagccgtagcgccatcagctacctgttaaaaaatggctgcaaacttactgacaaagaagaagattcagaaaaatttgctaaacgtcgtcgtcaagttgaaatccaaattcaaaggcttaccgaaaagttaataagtcggatgcctaaaggtcgagatttgaccaatgctaaatggttggagacactcttgactgctacaaccactgttgctgaagacaacgcccaagccaaacgctggcaggatattctgttaactcgatcaagttctctcccattcccccttgtttttgaaaccaacgaggatatggtttggtcaaagaatcaaaagggtaggctgtgtgttcacttcaatggcttaagcgatttaatttttgaggtgtactgcggcaatcgtcaacttcactggtttcaacgcttcctagaagaccaacagactaaacgcaaaagcaaaaatcagcattctagcggcttgttcacactcagaaatggtcatctagtttggcttgaaggtgagggtaaaggggaaccttggaatcttcaccacttgaccctttactgctgtgttgacaatcgcttgtggacagaggagggaacagaaatcgttcgccaagagaaagcagatgaaattactaaattcatcacaaacatgaagaagaaaagcgatctaagcgatacacagcaagctttgattcaacgtaaacaatcaacacttactcgaataaacaattcctttgagcgtcctagccaacccctttatcaaggtcaatcacacattttggttggagtaagcctgggactagaaaaacctgccacagtagcagtagtagatgcgatcgccaacaaagtcttggcttaccggagtattaaacaattacttggcgacaattacgaactgctaaatcgccagagacgacaacagcagtacctatctcacgaacgccacaaagcacaaaaaaacttctctcccaatcaatttggagcatctgagttagggcaacatatagacagattattagctaaagcaattgtagcgttagcgagaacctacaaagctggcagtattgtcttgcccaagttaggggatatgcgggaggttgtccaaagtgaaattcaagctatagcagaacaaaaatttcccggttatattgaaggtcagcaaaaatatgccaaacagtaccgggttaatgttcatcggtggagctacggcagattaattcaaagcattcaaagtaaagcagctcaaacaggaattgtgattgaggagggaaaacaacctattcgaggtagtccccacgacaaagcaaaggaattagcactttctgcttacaatctccgcctaactaggcgaagttaa

SEQ ID NO: 6 - DNA, 34 bp, synthetic construct
  source 1..34: mol_type = other DNA
tgacagctagctcagtcctaggtataatgctagc

SEQ ID NO: 7 - DNA, 24 bp, synthetic construct
  source 1..24: mol_type = other DNA
acgatgcgcccatctacaccaacg

SEQ ID NO: 8 - DNA, 24 bp, synthetic construct
  source 1..24: mol_type = other DNA
acggccaggacagtcgtttgccgt

SEQ ID NO: 9 - DNA, 24 bp, synthetic construct
  source 1..24: mol_type = other DNA
atctggaagatcaggatatgtggc

SEQ ID NO: 10 - DNA, 24 bp, synthetic construct
  source 1..24: mol_type = other DNA
tgacgcgatcggcataaccaccac

SEQ ID NO: 11 - DNA, 24 bp, synthetic construct
  source 1..24: mol_type = other DNA
gaagagcgagatctcgagctcttc

SEQ ID NO: 12 - DNA, 861 bp, synthetic construct
  source 1..861: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 13 - DNA, 589 bp, synthetic construct
  source 1..589: mol_type = other DNA
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 14 - DNA, 210 bp, synthetic construct
  source 1..210: mol_type = other DNA
tgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactga

SEQ ID NO: 15 - DNA, 795 bp, synthetic construct
  source 1..795: mol_type = other DNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 16 - DNA, 212 bp, synthetic construct
  source 1..212: mol_type = other DNA
gcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtaca

SEQ ID NO: 17 - DNA, 357 bp, synthetic construct
  source 1..357: mol_type = other DNA
tgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacttgagagcttagtacgtgaaacatgagagcttagtacgtac

SEQ ID NO: 18 - DNA, 29 bp, synthetic construct
  source 1..29: mol_type = other DNA
ttgacagcttatcatcgataagctttaat

SEQ ID NO: 19 - DNA, 19 bp, synthetic construct
  source 1..19: mol_type = other DNA
taatacgactcactatagg

SEQ ID NO: 20 - DNA, 29 bp, synthetic construct
  source 1..29: mol_type = other DNA
ttgacaattaatcatcggctcgtataatg

SEQ ID NO: 21 - DNA, 30 bp, synthetic construct
  source 1..30: mol_type = other DNA
ggctagctcagtcctaggtattgtgctagc

SEQ ID NO: 22 - DNA, 23 bp, synthetic construct
  source 1..23: mol_type = other DNA
ttgtcagatattacgcctgtgtg

SEQ ID NO: 23 - DNA, 24 bp, synthetic construct
  source 1..24: mol_type = other DNA
tcactcaagagcaagtagacaaaa

SEQ ID NO: 24 - DNA, 24 bp, synthetic construct
  source 1..24: mol_type = other DNA
aaacgcatctggggagtcacaggc

SEQ ID NO: 25 - DNA, 792 bp, synthetic construct
  source 1..792: mol_type = other DNA
atgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataa

SEQ ID NO: 26 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 27 - DNA, 1695 bp, synthetic construct
  source 1..1695: mol_type = other DNA
tcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaac

SEQ ID NO: 28 - DNA, 6833 bp, synthetic construct
  source 1..6833: mol_type = other DNA
ttgacagctagctcagtcctaggtataatactagtagggatcagcataataatacgttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttaattctaaagatctttgacagctagctcagtcctaggtataatactagtgcgatttaccgaccttttgcgttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggatctttgacagctagctcagtcctaggtataatactagtgtactgttttgtgctataaagttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggatctttgacagctagctcagtcctaggtataatactagtggctaacttttcattaagctgttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggatccttactcgagtctagactgcagtggatcccatggtacgcggccagatatcgacgtcttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcgttgatagagttattttaccactccctatcagtgatagagaaaagaattcaaaagatctaaagaggagaaaggatctatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 29 - DNA, 3414 bp, synthetic construct
  source 1..3414: mol_type = other DNA
atgaaaaaactactcgtcgccaatcgtggagaaatcgccgttcgtgtctttcgtgcctgtaatgaactcggactttctactgtagccgtctatgcaagagaagatgaatattctgttcatcgctttaaagcagatgaatcttatctaatcggtcaaggaaaaaaaccaattgatgcctatttggatattgatgatatcattcgtgttgcccttgaatcaggagcagatgctattcacccaggttatggcctcttatctgaaaatctcgaatttgctaccaaagttcgcgcagcaggtttagtttttgtgggtcctgaacttcatcatttggatattttcggtgataaaattaaagctaaagcagctgccgacgaagcgaaagttccaggaattcccggaacaaatggagcagtagatatcgacggcgctttagaatttgctaaaacttacggttatccagttatgattaaggccgctttaggcggtggtggtcgcggaatgcgagttgcgcgtaatgatgctgaaatgcatgatggttatgctagagcaaaatcagaagcaattggagcttttggttctggcgaaatctatgttgaaaaatatattgaaaatccaaaacatattgaagtccaaattcttggagataatcatggaaatattattcacctccatgagcgtgactgttcagtacaacgacgtaatcaaaaagtcattgaaattgcgccagccgtgggactttcactggacttccgtaacgaaatatgtgaagcagcagtcaaactttgtaaaaatgttggctatgttaatgctggtacagttgagtttcttgttaaagatgataaattttatttcattgaagtaaatccacgtgttcaagttgaacatacaattacggaacttatcacaggtgtagatattgtccaagctcaaattttgattgctcaaggtaaagacttacatagagaaattggacttccagcacagtctgaaattccacttttgggttcagcaattcaatgtcgaatcacaacagaagatccacaaaatggattcttgccagacacaggtaaaattgatacttaccgttcaccaggtggttttggtgtacgtttagatgtcggaaatgcctatgctggatatgaagtaactccatattttgattcacttttagtaaaagtttgtacttttgccaatgaatttagtgacagcgtaagaaaaatggaccgcgttctccatgaattccgtattcgtggtgtcaaaacaaatattcctttcttgattaatgttattgctaatgaaaacttcacaagtggtcaagcaacaacaacctttattgataatacaccaagtctgttcaacttcccacacttacgtgaccgtggaacaaagacgctgcattatttatcaatgattacagtcaatggcttcccagggattgaaaatactgaaaaacgtcattttgaagaaccacgtcaaccccttcttaatcttgaaaagaaaaagacagccaaaaatatcttagatgaacaaggtgctgatgctgttgttgattatgttaaaaatacaaaagaagtattattaacagatacaactttacgtgatgctcaccaatccttgcttgctactcgcttacgtttacaagatatgaaaggaattgcccaagcgattgaccaagggctaccagaacttttctcagctgagatgtggggtggtgcaacctttgatgtggcttaccgtttcttgaatgaatcaccgtggtatcgtctacgtaaactacgtaaactcatgccaaataccatgttccaaatgctcttccgtggttcaaatgcagttggttaccaaaactatccagataatgtgattgaagaatttatccgagtagcggcgcatgaaggtatcgatgttttccgtatctttgatagtcttaactggttaccccaaatggaaaaatccattcaagccgttcgagataatgggaaaattgcagaagcaacaatttgttatacgggagatatcctagacccaagccgaccaaaatataatattcaatactataaagacttagcaaaagaattagaagcaacaggtgctcatatcttggcagttaaagatatggcaggtttattgaaacctcaagctgcttatcgtttgatttcagaattaaaagataccgttgatttgccaattcatttgcatacacacgatacttcaggaaatggaattattacatattctggtgcaactcaagcaggtgtagacattattgacgtagcaacagcgagtcttgctggaggaacatctcaaccatcaatgcaatctatttactatgcacttgaacatggtcctcgccatgcctcaattaatgtaaaaaatgctgaacaaattgaccattattgggaagatgttcgtaaatattatgcaccatttgaagcgggtattacaagtcctcagacagaagtttacatgcatgaaatgcctggtggtcaatacactaacctaaaatctcaggcagcagcagttggtcttggacatcgttttgatgaaatcaaacaaatgtatcgtaaagttaacatgatgtttggagatattattaaagtaactccttcttcaaaagtagttggagatatggctctctttatgattcaaaatgatttaacagaagaagatgtttatgcgcgtggtaacgaacttaatttcccagaatctgtagtatcattcttccgtggagatttaggacaaccagtgggtggcttcccagaagaattacaaaaaattatcgtgaaagataaagccgtaattactgaccgtccaggcttacacgcagaaaaagttgattttgaaactgttaaagcagacctagaacaaaaaattggctatgaaccaggagatcacgaagttatctcatatatcatgtatccacaagttttccttgattatcaaaaaatgcaaagagaatttggagcagttacgctacttgacactcctactttcttacatggaatgcgtctcaatgaaaaaattgaagtacaaattgaaaaaggtaaaacactcagcattcgtttagatgaaataggagaacctgacctagctggaaatcgtgttctcttctttaacctaaatggtcaacgtcgagaagtcgtaattaatgaccaatctgtacaaactcaagttgtggctaaacgcaaagccgaaacaggaaatccaaaccaaattggagctactatgccaggttctgttcttgaaattttagttaaagctggtgacaaagttcaaaaaggtcaagccttgatggttactgaagccatgaaaatggaaacaactattgaagcaccatttgatggcgagattgttgaccttcatgtggttaaaggtgaggcaattcaaactcaagacttgttaatcgaaattaattaa

SEQ ID NO: 30 - DNA, 24 bp, synthetic construct
  source 1..24: mol_type = other DNA
gtaaacagcgcggcgtacgacctt

SEQ ID NO: 31 - DNA, 660 bp, synthetic construct
  source 1..660: mol_type = other DNA
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 32 - DNA, 546 bp, synthetic construct
  source 1..546: mol_type = other DNA
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaa

SEQ ID NO: 33 - DNA, 584 bp, synthetic construct
  source 1..584: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 34 - DNA, 711 bp, synthetic construct
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 35 - DNA, 32 bp, synthetic construct
  source 1..32: mol_type = other DNA
gcctaatgagtgagctaacttacattaattgc

SEQ ID NO: 36 - DNA, 230 bp, synthetic construct
  source 1..230: mol_type = other DNA
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctaaatgggttgaaag

(19) United States
(12) Patent Application Publication
(10) Pub. No.: US 2026/0035708 A1
(43) Pub. Date: Feb. 5, 2026

(54) Gene Editing System of Escherichia Coli and Gene Editing Method Thereof

(71) Applicant: National Tsing Hua University, Hsinchu (TW)

(72) Inventors: Yu-Chen Hu, Hsinchu (TW); Chin-Wei Chang, Hsinchu (TW); Jing-Wen Huang, Hsinchu (TW); June-Yen Chou, Kaohsiung (TW)

(21) Appl. No.: 19/363,998

(22) Filed: Oct. 21, 2025

Related U.S. Application Data
(62) Division of application No. 17/881,213, filed on Aug. 4, 2022.

(30) Foreign Application Priority Data
Jan. 26, 2022 (TW) 111103228

(51) Int. Cl. C12N 15/70; C12N 9/12; C12N 9/22

(52) U.S. Cl. CPC C12N 15/70; C12N 9/1241; C12N 9/22; C12N 2310/20; C12N 2800/40; C12N 2800/90

(57) ABSTRACT

A gene editing system of Escherichia coli includes an Escherichia coli, a helper plasmid and a donor plasmid. The helper plasmid successively includes a transposase complex expression cassette, a Cas12k expression cassette, a first sgRNA cassette, a first antibiotic resistance gene and a first replication origin. The donor plasmid successively includes a left end sequence of a ShCAST transposon, an exogenous gene expression cassette, a right end sequence of the ShCAST transposon, a second sgRNA cassette, a second antibiotic resistance gene and a second replication origin.

### CROSS-REFERENCE TO RELATED APPLICATION

This application is a divisional of U.S. patent application Ser. No. 17/881,213, filed Aug. 4, 2022, which claims priority to Taiwanese Application Number 111103228, filed Jan. 26, 2022, the disclosures of which are hereby incorporated by reference in their entirety.

### SEQUENCE LISTING

The sequence listing submitted via Patent Center, in compliance with 37 CFR § 1.52(e)(5), is incorporated herein by reference. The sequence listing XML file submitted via Patent Center contains the file 2505761.xml, created on Oct. 21, 2025, which is 56,674 bytes in size.

### BACKGROUND OF THE INVENTION

### Field of the Invention

The present disclosure relates to a gene editing technology for a microorganism. More particularly, the present disclosure relates to a gene editing system of Escherichia coli and a gene editing method thereof.

### Description of Related Art

Escherichia coli is widely used in industry for producing recombinant proteins and bio-derived chemicals. There are many different strains of Escherichia coli used for various purposes. For instance, BL21(DE3) strain is commonly used for recombinant protein production; MG1655, W3110 and W strains are used for producing bio-derived chemicals. DH10Bac strain contains a bacmid that combines the characteristics of the baculovirus genome and plasmid, and is used for bacmid engineering in the Bac-to-Bac™ system. Genetically modified bacmid is used for generating insect baculovirus to produce recombinant baculoviruses, which is used for recombinant protein production and gene delivery.

To improve the production performance of Escherichia coli, chromosomal integration of multiple metabolic pathway genes into the target chromosome to permanently manipulate the metabolic pathways is required. However, integration of large DNA cargo into Escherichia coli remains challenging. A-Red-based recombineering is common for DNA integration into Escherichia coli, but its payload capacity is limited. Since the advent of CRISPR/Cas9 technology, the combination of CRISPR/Cas9 and λ-Red further increases the size of the integrated DNA, which successfully increases the cargo size to be integrated into Escherichia coli genome to 10kb. Although CRISPR-Cas9/λ-Red system is leveraged in MG1655 strain for genetic engineering and metabolic engineering, CRISPR-induced double-strand break (DSB) may trigger genome instability and the integration efficiency is relatively low in other important Escherichia coli strains such as BL21(DE3) strain and W strain, presumably due to DNA repair ability for inserting the intended DNA into the genome. However, the DNA repair abilities of different strains vary widely, thus resulting in lower integration efficiencies in other Escherichia coli strains.

CRISPR-associated transposase (ShCAST) is an emerging powerful tool for on-target DNA transposition into Escherichia coli without inducing DSB, which combines the efficient DNA integration of transposases and CRISPR-mediated programmable DNA DBS free gene integration, but ShCAST technology still has the problem of poor integration at specific chromosomal positions. Therefore, how to improve the shortcomings of the above-mentioned gene editing system applied to Escherichia coli is one of the important issues to be solved at present.

### SUMMARY OF THE INVENTION

According to one aspect of the present disclosure, a gene editing system of Escherichia coli is provided. The gene editing system of Escherichia coli includes an Escherichia coli, a helper plasmid and a donor plasmid. The helper plasmid successively includes a transposase complex expression cassette, a Cas12k expression cassette, a first sgRNA cassette, a first antibiotic resistance gene and a first replication origin. The transposase complex expression cassette includes a first promoter, a tnsB gene, a tnsC gene and a tniQ gene, the Cas12k expression cassette includes a second promoter and a Cas12k gene, the first sgRNA cassette includes a third promoter and a sgRNA, and the sgRNA is composed of a scaffold and a spacer. The donor plasmid successively includes a left end sequence of a ShCAST transposon, an exogenous gene expression cassette, a right end sequence of the ShCAST transposon, a second sgRNA cassette, a second antibiotic resistance gene and a second replication origin. The exogenous gene expression cassette includes an exogenous gene, and the second sgRNA cassette includes a fourth promoter and the sgRNA. A sequence of the spacer is homologous to a first specific sequence of a chromosome of the Escherichia coli, and the first antibiotic resistance gene and the second antibiotic resistance gene are different.

According to another aspect of the present disclosure, a gene editing method of Escherichia coli includes steps as follows. A helper plasmid is constructed. The helper plasmid successively includes a transposase complex expression cassette, a Cas12k expression cassette, a first sgRNA cassette, a first antibiotic resistance gene and a first replication origin. The transposase complex expression cassette includes a first promoter, a tnsB gene, a tnsC gene and a tniQ gene, the Cas12k expression cassette includes a second promoter and a Cas12k gene, the first sgRNA cassette includes a third promoter and a sgRNA, and the sgRNA is composed of a scaffold and a spacer. A donor plasmid is constructed. The donor plasmid successively includes a left end sequence of a ShCAST transposon, an exogenous gene expression cassette, a right end sequence of the ShCAST transposon, a second sgRNA cassette, a second antibiotic resistance gene and a second replication origin. The exogenous gene expression cassette includes an exogenous gene, and the second sgRNA cassette includes a fourth promoter and the sgRNA, and the first antibiotic resistance gene and the second antibiotic resistance gene are different. The helper plasmid and the donor plasmid are co-transformed into an Escherichia coli to obtain a transformant. The transformant is cultured for an editing time at an editing temperature, in which the helper plasmid expresses a TnsB protein, a TnsC protein, a TniQ protein and a Cas12k protein to form a ShCAST transposon protease complex, the helper plasmid expresses the sgRNA, the donor plasmid expresses the sgRNA, and the exogenous gene is inserted into a first specific sequence of the transformant.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIG. 1A is a schematic view showing a construction of a helper plasmid according to one embodiment of the present disclosure.

FIG. 1B is a schematic view showing a construction of a donor plasmid according to one embodiment of the present disclosure.

FIG. 2 is a flow diagram showing a gene editing method of Escherichia coli according to one embodiment of the present disclosure.

FIG. 3A is a schematic view showing constructions of a donor plasmid and helper plasmids of Test Examples.

FIG. 3B is a schematic view showing integration efficiencies of Test Examples using blue-white screening.

FIG. 3C shows analytical results of blue-white screening of Test Examples targeting different lacZ gene positions.

FIG. 3D shows analytical results of the on-target efficiency of Test Examples at different lacZ gene positions.

FIG. 3E shows analytical results of the on-target efficiency of Test Examples in different Escherichia coli strains.

FIG. 4A is a schematic view showing constructions of helper plasmids of first embodiment of the present disclosure and a helper plasmid of Test Example.

FIG. 4B is a schematic view showing constructions of helper plasmids and a donor plasmid of first embodiment of the present disclosure.

FIG. 4C shows analytical results of relative Cas12k gene expression of a gene editing system of Escherichia coli according to first embodiment of the present disclosure.

FIG. 4D shows analytical results of the on-target efficiency into lacZ gene of the gene editing system of Escherichia coli according to first embodiment of the present disclosure.

FIG. 4E shows analytical results of the on-target efficiency of the gene editing system of Escherichia coli at different genomic sites according to first embodiment of the present disclosure.

FIG. 4F shows analytical results of the on-target efficiency of the gene editing system of Escherichia coli at different editing temperatures according to first embodiment of the present disclosure.

FIG. 4G shows analytical results of whole genome sequencing analyses of off-target integration of the gene editing system of Escherichia coli and the gene editing method of Escherichia coli according to the first embodiment of the present disclosure.

FIG. 5A is a schematic view showing constructions of a helper plasmid and donor plasmids of second embodiment of the present disclosure.

FIG. 5B shows photographs of colony formation after being processed by a gene editing system of Escherichia coli according to the second embodiment of the present disclosure.

FIG. 5C is a statistical graph of the number of successfully edited colonies of the gene editing system of Escherichia coli at different stuffer DNA sizes according to second embodiment of the present disclosure.

FIG. 5D shows analytical results of the on-target efficiency of the gene editing system of Escherichia coli at different stuffer DNA sizes according to second embodiment of the present disclosure.

FIG. 6A is a schematic view showing constructions of a helper plasmid and a donor plasmid of third embodiment of the present disclosure.

FIG. 6B is a schematic view showing a low-acid-producing platform constructed by a gene editing system of Escherichia coli according to third embodiment of the present disclosure.

FIG. 6C shows the expression analysis of the acid production-related genes of the gene editing system of Escherichia coli according to third embodiment of the present disclosure.

FIGS. 6D, 6E and 6F show the acid production analysis of the gene editing system of Escherichia coli according to third embodiment of the present disclosure.

FIG. 6G is a fluorescence image of a fermentation broth of the gene editing system of Escherichia coli according to third embodiment of the present disclosure.

FIG. 6H shows quantitative analytical results of fluorescence value of the gene editing system of Escherichia coli according to third embodiment of the present disclosure.

FIG. 7A is a schematic view showing constructions of a helper plasmid and a donor plasmid of fourth embodiment of the present disclosure.

FIG. 7B is an editing schematic view of a gene editing system of Escherichia coli according to fourth embodiment of the present disclosure.

FIG. 7C shows analytical results of relative pyc gene expression of the gene editing system of Escherichia coli according to fourth embodiment of the present disclosure.

FIG. 7D shows analytical results of relative adhE gene expression of the gene editing system of Escherichia coli according to fourth embodiment of the present disclosure.

FIG. 7E shows analytical results of succinate production of the gene editing system of Escherichia coli according to fourth embodiment of the present disclosure.

FIG. 8A is a schematic view showing constructions of a helper plasmid and a donor plasmid of fifth embodiment of the present disclosure.

FIG. 8B is a flowchart of the baculovirus editing by a gene editing system of Escherichia coli according to fifth embodiment of the present disclosure.

FIG. 8C shows analytical results of verifying the successful integration of the gene editing system of Escherichia coli according to fifth embodiment of the present disclosure by colony PCR.

FIG. 8D shows analytical results of the expression of the V-cath gene to be knocked out by the gene editing system of Escherichia coli according to fifth embodiment of the present disclosure.

FIG. 8E shows analytical results of HEK293-FT transduced by recombinant baculovirus after being processed by the gene editing system of Escherichia coli according to fifth embodiment of the present disclosure.

### DESCRIPTION OF THE INVENTION

### Gene Editing System of Escherichia coli

A gene editing system of Escherichia coli includes an Escherichia coli (not shown), a helper plasmid 100 and a donor plasmid 300. The Escherichia coli can be BL21(DE3) strain, BW25113 strain, MG1655 strain, W3110 strain, W strain or DH10Bac strain.

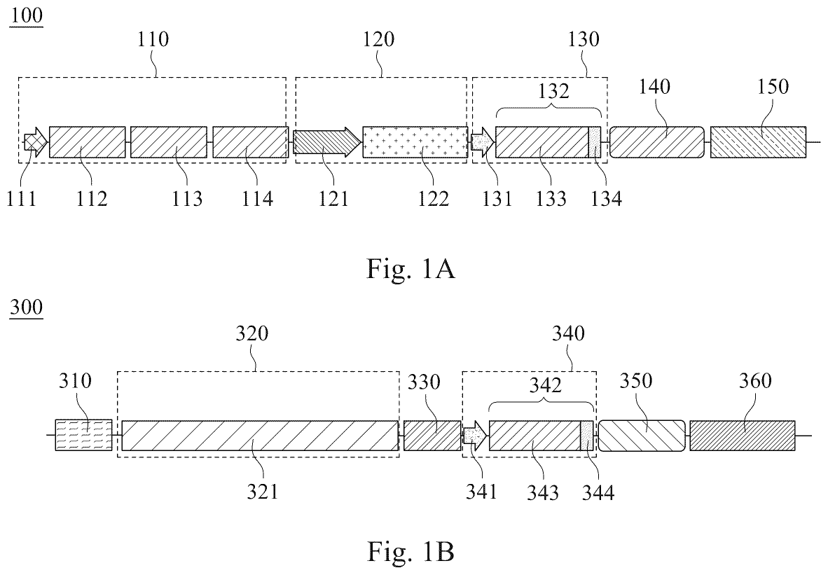

Please refer to FIG. 1A, which is a schematic view showing a construction of a helper plasmid 100 according to one embodiment of the present disclosure. The helper plasmid 100 successively includes a transposase complex expression cassette 110, a Cas12k expression cassette 120, a first sgRNA cassette 130, a first antibiotic resistance gene 140 and a first replication origin 150. The transposase complex expression cassette 110 includes a first promoter 111, a tnsB gene 112, a tnsC gene 113 and a tniQ gene 114. The Cas12k expression cassette 120 includes a second promoter 121 and a Cas12k gene 122. The first sgRNA cassette 130 includes a third promoter 131 and a sgRNA 132. The sgRNA 132 is composed of a scaffold 133 and a spacer 134. A sequence of the spacer 134 is homologous to a first specific sequence of a chromosome of the Escherichia coli. In detail, the transposase complex expression cassette 110 can further include a terminator (not shown), and the terminator is connected to the 3′ end of the tniQ gene 114. The second promoter 121 can be a lac promoter, a Tet promoter, a T7 promoter, a Tac promoter or a J23118 promoter.

Please refer to FIG. 1B, which is a schematic view showing a construction of a donor plasmid 300 according to one embodiment of the present disclosure. The donor plasmid 300 successively includes a left end sequence of a ShCAST transposon 310, an exogenous gene expression cassette 320, a right end sequence of the ShCAST transposon 330, a second sgRNA cassette 340, a second antibiotic resistance gene 350 and a second replication origin 360. The exogenous gene expression cassette 320 includes an exogenous gene 321, and the second sgRNA cassette 340 includes a fourth promoter 341 and the sgRNA 342. The sgRNA 342 is composed of the scaffold 343 and the spacer 344, wherein the sequence of the sgRNA 342 is same as the sequence of the sgRNA 132, and the sequence of the spacer 344 is homologous to the first specific sequence of the chromosome of the Escherichia coli. The first antibiotic resistance gene 140 and the second antibiotic resistance gene 350 are different. In detail, the exogenous gene expression cassette 320 can further include a fifth promoter (not shown) and a third antibiotic resistance gene (not shown), and the third antibiotic resistance gene is different from the first antibiotic resistance gene 140 and the second antibiotic resistance gene 350. In addition, the donor plasmid 300 can further include a CRISPRi module (not shown), and the CRISPRi module is located between the left end sequence of the ShCAST transposon 310 and the exogenous gene expression cassette 320.

### Gene Editing Method of Escherichia coli

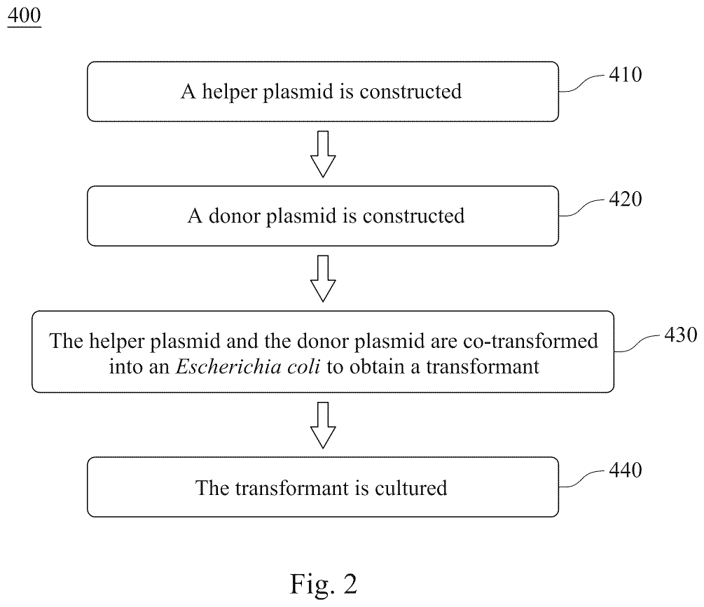

Please refer to FIG. 2, which is a flow diagram showing a gene editing method of Escherichia coli 400 according to one embodiment of the present disclosure. The gene editing method of Escherichia coli 400 includes Step 410, Step 420, Step 430 and Step 440.

In Step 410, a helper plasmid is constructed. The helper plasmid successively includes a transposase complex expression cassette, a Cas12k expression cassette, a first sgRNA cassette, a first antibiotic resistance gene and a first replication origin. The transposase complex expression cassette includes a first promoter, a tnsB gene, a tnsC gene and a tniQ gene, the Cas12k expression cassette includes a second promoter and a Cas12k gene, the first sgRNA cassette includes a third promoter and a sgRNA, and the sgRNA is composed of a scaffold and a spacer.

In Step 420, a donor plasmid is constructed. The donor plasmid successively includes a left end sequence of a ShCAST transposon, an exogenous gene expression cassette, a right end sequence of the ShCAST transposon, a second sgRNA cassette, a second antibiotic resistance gene and a second replication origin. The exogenous gene expression cassette includes an exogenous gene, and the second sgRNA cassette includes a fourth promoter and the sgRNA, and the first antibiotic resistance gene and the second antibiotic resistance gene are different.

In Step 430, the helper plasmid and the donor plasmid are co-transformed into an Escherichia coli to obtain a transformant. In detail, a selection step can be further included, wherein the transformant is cultured in a medium containing an antibiotic to select the transformant that is successfully co-transformed the helper plasmid and the donor plasmid, and the antibiotic is an ampicillin (Amp), a kanamycin (Km), a spectinomycin (Spc) or a chloramphenicol (Cm).

In Step 440, the transformant is cultured for an editing time at an editing temperature, in which the helper plasmid expresses a TnsB protein, a TnsC protein, a TniQ protein and a Cas12k protein to form a ShCAST transposon protease complex, the helper plasmid expresses the sgRNA, the donor plasmid expresses the sgRNA, and the exogenous gene is inserted into a first specific sequence of the transformant.

Reference will now be made in detail to the present embodiments of the present disclosure, examples of which are illustrated in the accompanying drawings.

### 1. Validation that the ShCAST System can Integrate DNA into a Designated Position in a Variety of Escherichia coli Strains

To evaluate whether ShCAST functioned in common Escherichia coli strains, a donor plasmid and 5 helper plasmids are constructed according to the conventional ShCAST system as Test Examples.

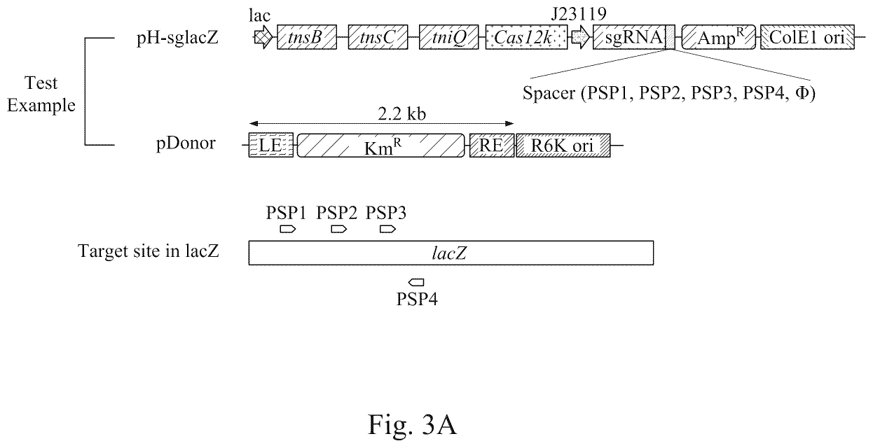

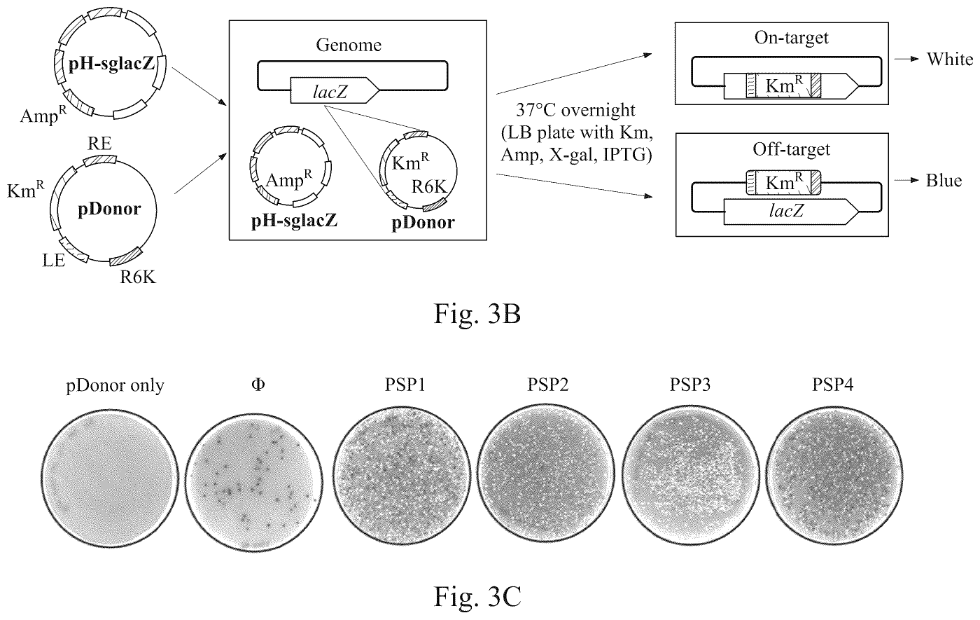

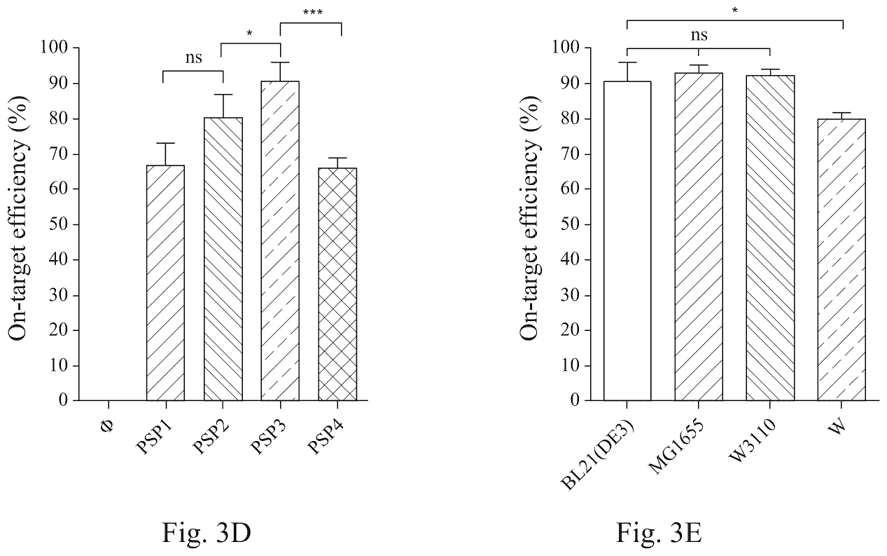

Please refer to FIG. 3A, which is a schematic view showing constructions of a donor plasmid and helper plasmids of Test Examples. The helper plasmid (pH-sglacZ) of Test Example successively includes the tnsB gene with the sequence referenced as SEQ ID NO: 2, the tnsC gene with the sequence referenced as SEQ ID NO: 3, the tniQ gene with the sequence referenced as SEQ ID NO: 4 and the Cas12k gene with the sequence referenced as SEQ ID NO: 5 driven by the lac promoter with the sequence referenced as SEQ ID NO: 1, a sgRNA driven by the J23119 promoter with the sequence referenced as SEQ ID NO: 6, an ampicillin resistance gene (Amp^{R}) with the sequence referenced as SEQ ID NO: 12, and a ColE1 replication origin (ori) with the sequence referenced as SEQ ID NO: 13. The sgRNA is composed of a scaffold and a spacer. The constructed helper plasmids differ in the spacer of the sgRNA, which can target 4 different protospacers (PSP1 to PSP4) in lacZ gene and target a scrambling sequence as a control group (Φ), and other part of these helper plasmids are the same. The sequence of the spacer targeting PSP1 is referenced as SEQ ID NO: 7, the sequence of the spacer targeting PSP2 is referenced as SEQ ID NO: 8, the sequence of the spacer targeting PSP3 is referenced as SEQ ID NO: 9, the sequence of the spacer targeting PSP4 is referenced as SEQ ID NO: 10, and the sequence of the spacer of the control group (Φ) is referenced as SEQ ID NO: 11.

The donor plasmid of Test Example (pDonor) successively includes the left end sequence of the ShCAST transposon with the sequence referenced as SEQ ID NO: 14, a kanamycin resistance gene (Km^{R}) with the sequence referenced as SEQ ID NO: 15, the right end sequence of the ShCAST transposon with the sequence referenced as SEQ ID NO: 16, and a R6K replication origin (ori) with the sequence referenced as SEQ ID NO: 17.

The BL21(DE3) strain is tested first, because the BL21(DE3) strain is difficult to edit using CRISPR/Cas9 or λ-Red systems. Please refer to FIG. 3B, which is a schematic view showing integration efficiencies of Test Examples using blue-white screening. The constructed pH-sglacZ and pDonor are co-electroporated into the BL21(DE3) strain, selected in an LB plate containing selective antibiotics, X-gal, and IPTG, and cultured at 37° C. overnight. Colonies only formed after the Km^{R} gene integration because the R6K ori does not support pDonor replication in pir gene-negative cells such as the BL21(DE3) strain, the MG1655 strain, the W3110 strain and the W strain. Therefore, white colonies represent on-target integration into the lacZ gene, and blue colonies represent off-target integration to other sites.

Please refer to FIG. 3C, which shows analytical results of blue-white screening of Test Examples targeting different lacZ gene positions. Electroporation of pDonor alone conferred no colony formation (i.e. no integration) while co-electroporation with the pDonor and the pH-sglacZ of control group resulted in scarce blue colonies (i.e. sporadic off-target integration). Co-electroporation of the pDonor and the pH-sglacZ targeting PSP1 to PSP4 conferred white colonies and blue colonies to different degrees.

Please refer to FIG. 3D, which shows analytical results of the on-target efficiency of Test Examples at different lacZ gene positions. The on-target efficiency is defined as the white colony forming units (cfu) divided by total cfu. In FIG. 3D, the selection of the site plays a key role in the on-target efficiency of Test Examples. The site with the worst on-target efficiency is PSP4 with the on-target efficiency about 66.0%, and the site with the best on-target efficiency is PSP3 with the on-target efficiency about 90.4%.

After the successful editing of the BL21(DE3) strain, other Escherichia coli strains commonly used in biotechnology are tested experimentally in a similar manner for on-target integration into PSP3 of the lacZ gene. Please refer to FIG. 3E, which shows analytical results of the on-target efficiency of Test Examples in different Escherichia coli strains. By calculating the proportion of white colonies, the results indicate that Test Example conferred high on-target efficiency into PSP3 in the BL21 (DE3) strain, the MG1655 strain and the W3110 strain with the on-target efficiency about 91.8±1.2%, and the on-target efficiency is also as high as about 79.8% in the W strain.

### 2. The Gene Editing System of Escherichia coli of the Present Disclosure Promotes the On-Target Efficiency

Despite the original ShCAST system (i.e., Test Example) can successfully integrate target gene into designated site on Escherichia coli chromosome, the editing efficiency of different positions is different, and the on-target efficiency is relatively low in some sites (e.g. PSP4 of the lacZ gene). When using the original ShCAST system to target specific genes, it is necessary to screen out sgRNAs with better editing efficiency, which is inconvenient to use. Therefore, the gene editing system of Escherichia coli of the present disclosure can optimize the ShCAST system by enhancing the expression of the Cas12k gene.

Precise integration requires concerted action of Cas12k protein and sgRNA to locate the protospacer adjacent motif (PAM) sequence and PSP. However, the Cas12k gene expressed at the end of operon of the ShCAST original system under the lac promoter, which might result in insufficient Cas12k protein expression and less effective integration. Therefore, the gene editing system of Escherichia coli of the present disclosure improves the original ShCAST system by constructing a series of helper plasmids (pH-promoter).

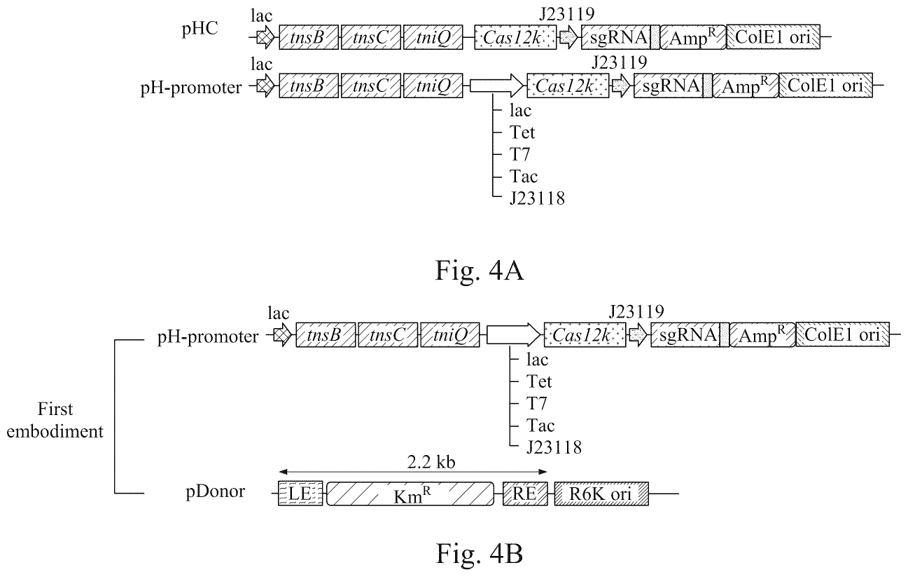

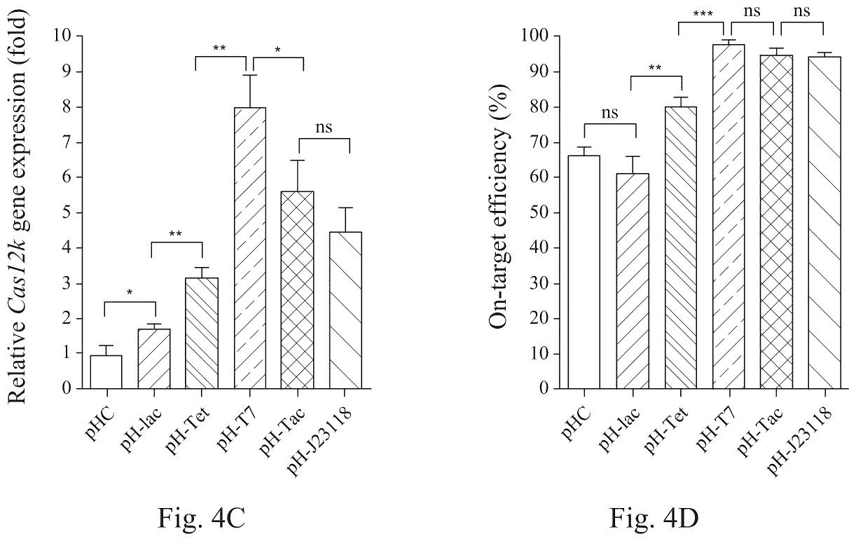

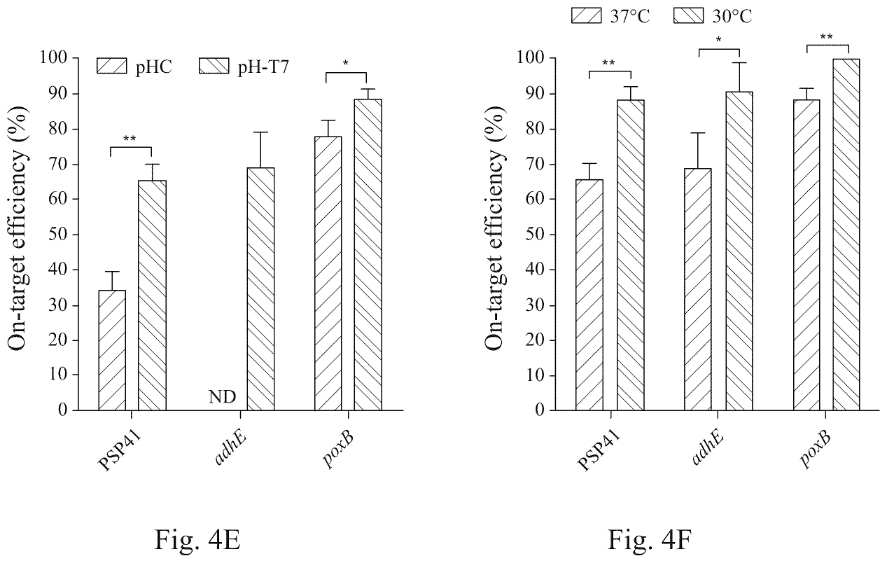

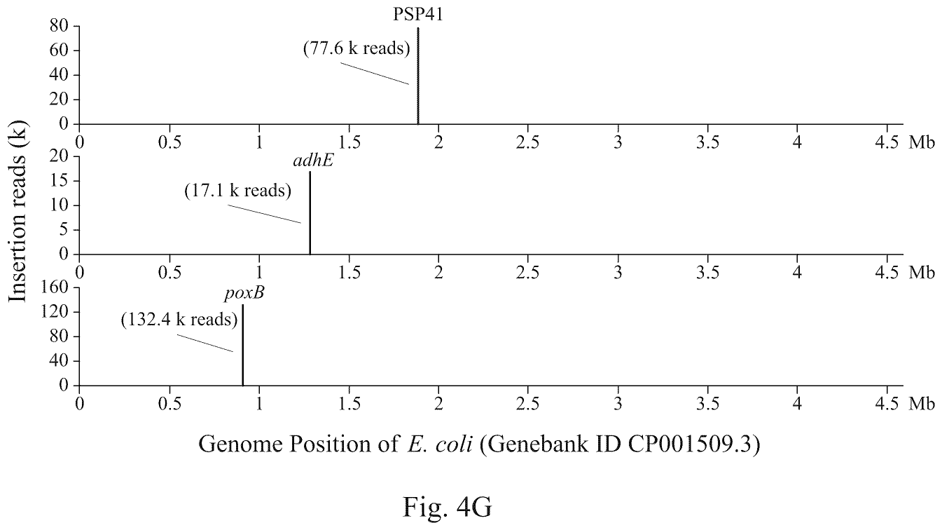

Please refer to FIGS. 4A and 4B. FIG. 4A is a schematic view showing constructions of helper plasmids (pH-promoter) of first embodiment of the present disclosure and the helper plasmid (pHC) of Test Example, wherein the construction of the pHC of Test Example is the same as the construction of the pH-sglacZ with the sgRNA targeting PSP4 in FIG. 3A, and will not be repeated here. FIG. 4B is a schematic view showing constructions of the helper plasmids and a donor plasmid (pDonor) of first embodiment of the present disclosure, wherein the pDonor of first embodiment of the present disclosure is same as the donor plasmid of Test Example, and will not be repeated here. The difference between the helper plasmids of the first embodiment of the present disclosure and the helper plasmid of Test Example is that the Cas12k gene is under another independent promoter to form a Cas12k expression cassette, and there is a terminator with the sequence referenced as SEQ ID NO: 35 after the tniQ gene. The different independent promoters used in the helper plasmids of first embodiment are respectively the lac promoter with the sequence referenced as SEQ ID NO: 1, a Tet promoter with the sequence referenced as SEQ ID NO: 18, a T7 promoter with the sequence referenced as SEQ ID NO: 19, a Tac promoter with the sequence referenced as SEQ ID NO: 20, and a J23118 promoter with the sequence referenced as SEQ ID NO: 21, and are named pH-lac, pH-Tet, pH-T7, pH-Tac, pH-J23118, respectively. The spacer of the sgRNA of the helper plasmids of the first embodiment and the spacers of the helper plasmids of Test Example are the same, with the sequence referenced as SEQ ID NO: 10, which target the most difficult-to-integrate position (PSP4) in the lacZ gene. The sequence of the scaffold of the sgRNA is referenced as SEQ ID NO: 36.

Please refer to FIG. 4C, which shows analytical results of relative Cas12k gene expression of the gene editing system of Escherichia coli according to first embodiment of the present disclosure. The constructed helper plasmids are respectively electroporated into the BL21(DE3) strain. After screening and culturing in a medium containing ampicillin (Amp) and induction with IPTG, RNA of the BL21(DE3) strain is extracted for analysis. The qRT-PCR analyses confirm that the Cas12k gene expression is the weakest in Test Example (pHC) and is enhanced by using an independent promoter, with the following strength order: T7>Tac≥J23118>Tet>lac.

Please refer to FIG. 4D, which shows analytical results of on-target efficiency into the lacZ gene of the gene editing system of Escherichia coli according to first embodiment of the present disclosure. To evaluate the effect of enhancing the Cas12k gene expression on gene editing efficiency, the constructed helper plasmids (the pH-lac, the pH-Tet, the pH-T7, the pH-Tac, the pH-J23118 or the pHC) are co-electroporated with the pDonor into the BL21(DE3) strain, followed by streaking onto LB plates containing Amp/Km/IPTG/X-gal. When using the T7 promoter, the Tac promoter and the J23118 promoter to independently express the Cas12k gene, the on-target efficiency can be increased to 94.2%-97.6%, while only about 66.0% in Test Example (the pHC). However, when the Cas12k gene expression exceeded the threshold, the on-target efficiency cannot continue to increase. The lac promoter does not increase the on-target efficiency, probably due to lower Cas12k gene expression.

To demonstrate the versatility of the gene editing system of Escherichia coli of the present disclosure, the pH-T7 and the pHC are further constructed with replaced sgRNA targeting other sites in the BL21(DE3) strain to compare the gene editing efficiency of the pH-T7 and the pHC at different sites. The targeting sites are PSP41, adhE gene and poxB gene, respectively. The sequence of the spacer targeting PSP41 is referenced as SEQ ID NO: 22, sequence of the spacer targeting the adhE gene is referenced as SEQ ID NO: 23, and the sequence of the spacer targeting the poxB gene is referenced as SEQ ID NO: 24.

Please refer to FIG. 4E, which shows analytical results of the on-target efficiency of the gene editing system of Escherichia coli at different genomic sites according to first embodiment of the present disclosure. In FIG. 4E, compared with Test Example, the gene editing system of Escherichia coli according to first embodiment of the present disclosure can improve the on-target efficiency at the site of PSP41 from 34.1% to 65.3%, the on-target efficiency at the site of the adhE gene can be increased from 0% to 68.7%, the on-target efficiency at the site of the poxB gene can be increased from 79.3% to 88.2%. The results demonstrate again that increasing the Cas12k gene expression can enhance the on-target efficiency.

In addition, the effect of editing temperature on gene editing is compared experimentally. The pH-T7 and the pDonor are co-electroporated into the BL21(DE3) strain to target the sites of PSP41, the adhE gene and the poxB gene, and overnight selection at 30° C. or 37° C. Please refer to FIGS. 4F and 4G, FIG. 4F shows analytical results of the on-target efficiency of the gene editing system of Escherichia coli at different editing temperatures according to first embodiment of the present disclosure, and FIG. 4G shows analytical results of whole genome sequencing analyses of off-target integration of the gene editing system of Escherichia coli and the gene editing method of Escherichia coli according to the first embodiment of the present disclosure. The results show that 30° C. conferred significantly higher on-target efficiency than 37° C. at the sites of PSP41, the adhE gene and the poxB gene, and the effect is significant (p<0.05). The gene editing method of Escherichia coli according to first embodiment of the present disclosure can achieve 100% on-target efficiency at the poxB gene and 90% on-target efficiency at the adhE gene. It is worth noting that the adhE gene is unable to edit using Test Example (i.e., the original ShCAST system). The analytical results of whole genome sequencing analyses of colonies for the 3 editing experiments in FIG. 4G confirm the absence of off-target integration.

To sum up, the gene editing system of Escherichia coli and the gene editing method of Escherichia coli of the present disclosure can enhance the Cas12k gene expression by using an independent strong promoter (such as the T7 promoter or the Tac promoter), and can further reduce the editing temperature to 30° C. to optimize the gene editing method of Escherichia coli of the present disclosure. Therefore, the gene editing system of Escherichia coli and the gene editing method of Escherichia coli of the present disclosure can efficiently, accurately and multiplely integrate target gene into the Escherichia coli genome to generate stable strains. Subsequent experiments further verify the applicability of the gene editing system of Escherichia coli and the gene editing method of Escherichia coli of the present disclosure.

### 3. The Gene Editing System of Escherichia coli of the Present Disclosure Enables High Efficiency Integration of DNA as Large as 14.5 kb

Bio-derived product production typically requires integration of a synthetic pathway, which often includes multiple genes and is difficult to integrate simultaneously using λ-Red system or CRISPR Cas9/λ-Red. In particular, CRISPR/Cas9-mediated integration is inefficient in the BL21(DE3) strain.

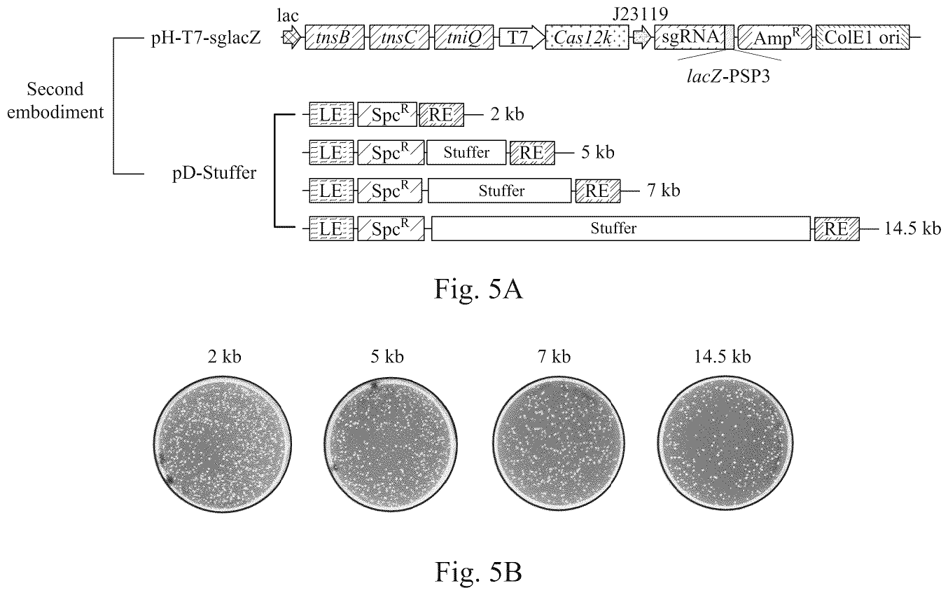

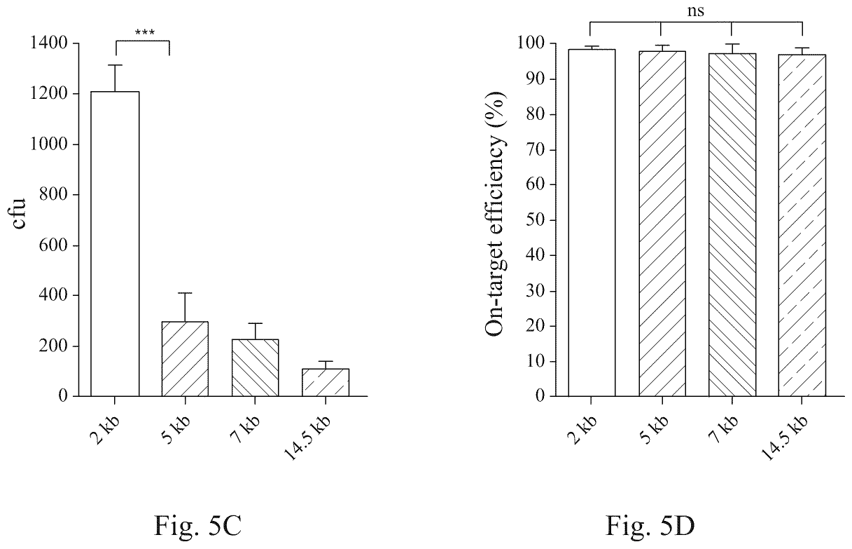

Please refer to FIG. 5A, which is a schematic view showing constructions of a helper plasmid (pH-T7-sglacZ) and donor plasmids (pD-Stuffer) of second embodiment of the present disclosure. To evaluate whether the gene editing system of Escherichia coli of the present disclosure allowed integration of large DNA into the BL21(DE3) strain, 4 pD-Stuffers carrying spectinomycin resistance gene (Spc^{R}) and stuffer DNA are constructed to test the DNA fragment size that the gene editing system of Escherichia coli of the present disclosure can be integrated into the target gene. The sequence of the Spc^{R} gene is referenced as SEQ ID NO: 25, and the size of the stuffer DNA is 2 kb, 5 kb, 7 kb and 14.5 kb, respectively. The pH-T7-sglacZ, the pH-T7 targeting PSP3, is used in the experiment. The pH-T7-sglacZ is co-electroporated with 4 pD-Stuffers carrying stuffer DNA of different lengths into the BL21 (DE3) strain, respectively. Incubate overnight with Spc/Amp/IPTG/X-gal at 30° C. for the blue-white screening.

Please refer to FIGS. 5B to 5D. FIG. 5B shows photographs of colony formation after being processed by a gene editing system of Escherichia coli according to the second embodiment of the present disclosure. FIG. 5C is a statistical graph of the number of successfully edited colonies of the gene editing system of Escherichia coli at different stuffer DNA sizes according to second embodiment of the present disclosure. FIG. 5D shows analytical results of the on-target efficiency of the gene editing system of Escherichia coli at different stuffer DNA sizes according to second embodiment of the present disclosure.

In FIG. 5B, whether gene editing is performed with the pD-Stuffer carrying the stuffer DNA size of 2 kb, 5 kb, 7 kb, or 14.5 kb, mostly white colonies are observed, indicating that the stuffer DNA is on-target integrated into the target lacZ gene, respectively. In FIG. 5C, the corresponding cfu gradually decreases with increasing the stuffer DNA size (from about 1200 cfu for 2 kb to about 100 cfu for 14.5 kb). However, the results in FIG. 5D show that the on-target efficiency are similarly high (>96.6%) for all 4 donor plasmid with different stuffer DNA sizes.

### 4. Genome and Metabolic Engineering of the BL21(DE3) Strain by the Gene Editing System of Escherichia coli of the Present Disclosure to Enhance Protein Production

The BL21(DE3) strain is widely used for protein production, but acid accumulation byproducts produced by bacteria during large-scale and high-density fermentation often reduces pH and product titer and quality (e.g., acid-sensitive proteins). Although knocking out genes contributing to the production of byproducts such as acetate (ackA gene, pta gene, YccX gene, poxB gene), formate (pflB gene) or lactate (ldhA gene, dld gene, ptsG gene) can enhance bio-derived product production, deletion of these genes leads to slower cell growth. In addition, since the deletion of the poxB gene can reduce the production of acetate, a low-acid-producing BL21(DE3) strain can be constructed by using the gene editing system of Escherichia coli of the present disclosure.

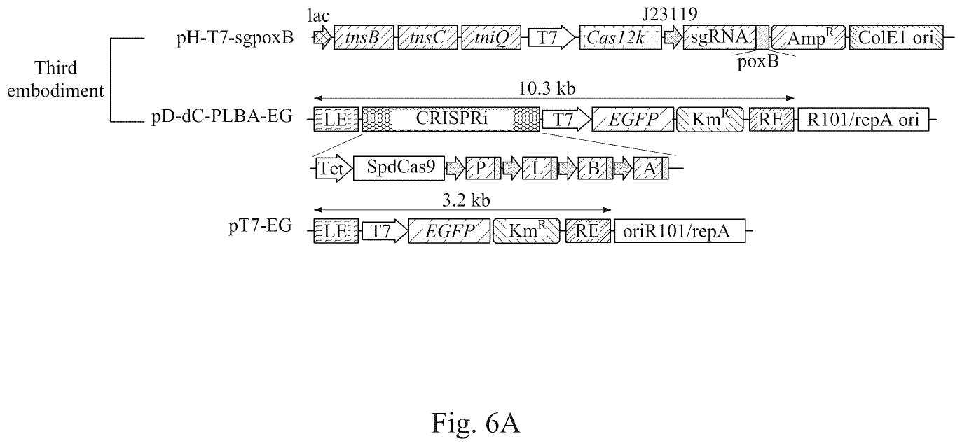

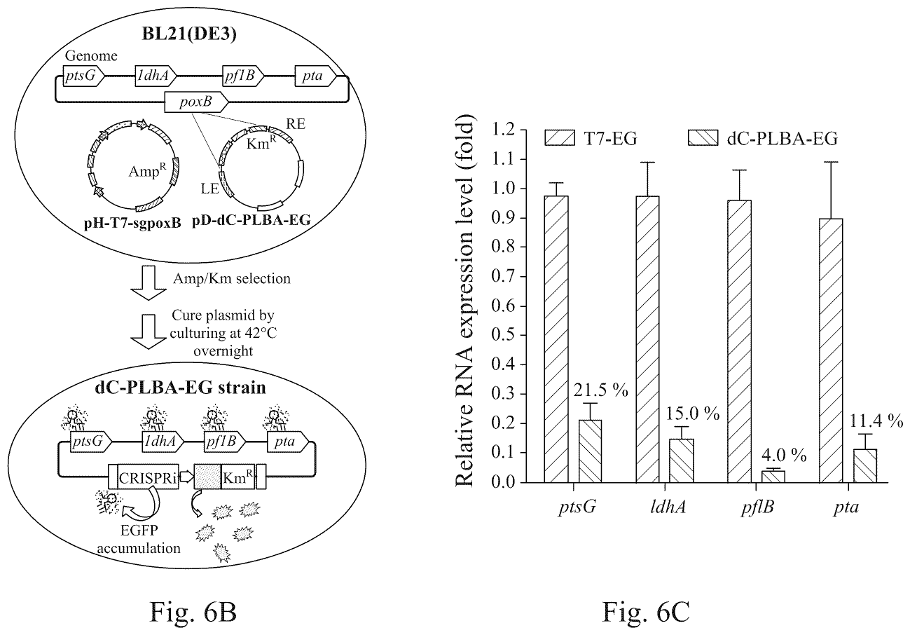

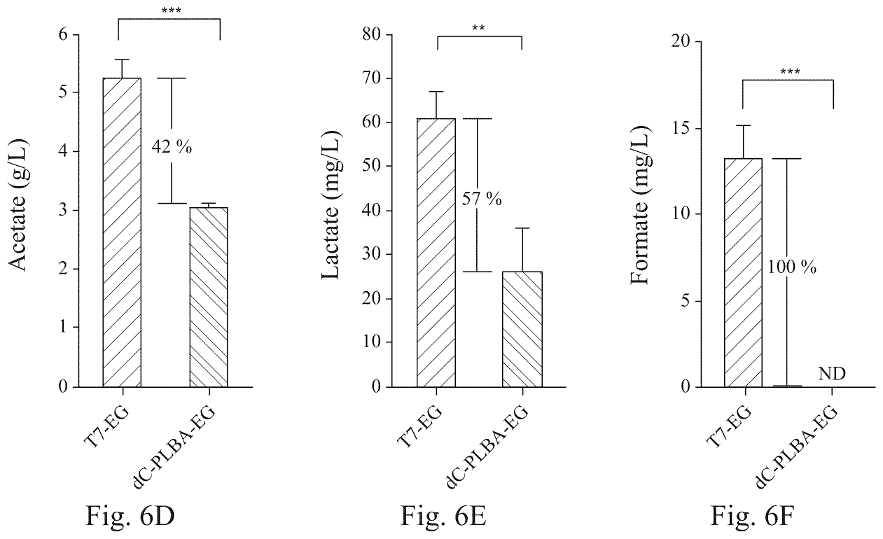

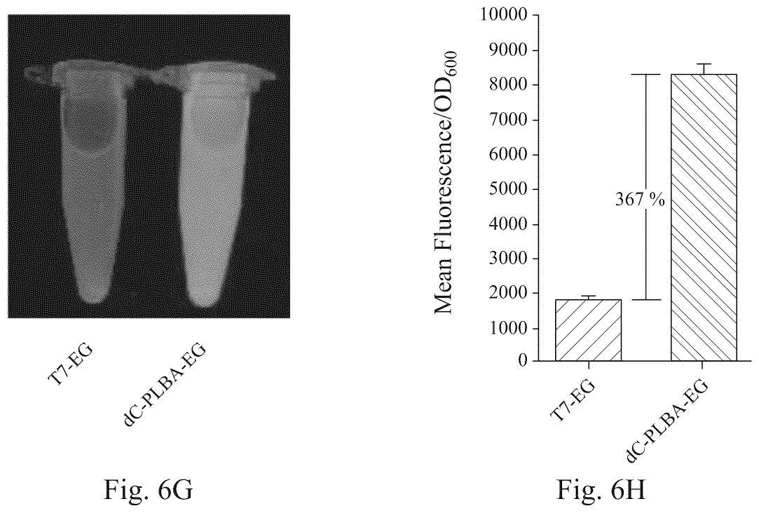

Please refer to FIG. 6A, which is a schematic view showing constructions of a helper plasmid (pH-T7-sgpoxB) and a donor plasmid (pD-dC-PLBA-EG) of third embodiment of the present disclosure. In the experiment, the gene editing system of Escherichia coli of the present disclosure is used to construct the low acid-producing BL21 (DE3) strain, and the pH-T7 is used as the backbone to construct the pH-T7-sgpoxB, which replaced the spacer with the spacer targeting the poxB gene (the sequence is reference as SEQ ID NO: 24). The pD-dC-PLBA-EG is simultaneously constructed, and the pD-dC-PLBA-EG successively includes the left end sequence of the ShCAST transposon with the sequence referenced as SEQ ID NO: 14, a CRISPRi module, the T7 promoter with the sequence referenced as SEQ ID NO: 19, EGFP gene with the sequence referenced as SEQ ID NO: 26, the Km^{R} gene with the sequence referenced as SEQ ID NO: 15, the right end sequence of the ShCAST transposon with the sequence referenced as SEQ ID NO: 16 and a R101/repA replication origin with the sequence referenced as SEQ ID NO: 27. The CRISPRi module can simultaneously inhibit the ptsG gene, the IdhA gene, the pflB gene and the pta gene, and the sequence of the CRISPRi module is referenced as SEQ ID NO: 28. In the experiment, a donor plasmid pT7-EG expressing only the EGFP gene and the Km^{R} gene is constructed as a control group, which does not include the CRISPRi module.

Please further refer to FIG. 6B, which is a schematic view showing a low-acid-producing platform constructed by a gene editing system of Escherichia coli according to third embodiment of the present disclosure. In the experiment, the helper plasmid (pH-T7-sgpoxB) and the donor plasmid (pD-dC-PLBA-EG) are co-electroporated into the BL21(DE3) strain, and a dC-PLBA-EG strain that expresses EGFP and CRISPRi module suppressing the ptsG gene (P), the IdhA gene (L), the pflB gene (B), and the pta gene (A) is generated. A control T7-EG strain is generated similarly to express EGFP but no CRISPRi module. It is confirmed by colony PCR and qRT-PCR that the gene editing system of Escherichia coli according to third embodiment of the present disclosure can on-target integrate the 10.3 kb expression cassette including CRISPRi module, EGFP gene and the Km^{R} gene and 3.7 kb expression cassette including the EGFP gene and the Km^{R} gene into the poxB gene and disrupted the poxB gene at the same time.

Please refer to FIGS. 6C to 6H. FIG. 6C shows the expression analysis of the acid production-related genes of the gene editing system of Escherichia coli according to third embodiment of the present disclosure. FIGS. 6D, 6E and 6F show the acid production analysis of the gene editing system of Escherichia coli according to third embodiment of the present disclosure. FIG. 6G is a fluorescence image of a fermentation broth of the gene editing system of Escherichia coli according to third embodiment of the present disclosure. FIG. 6H shows quantitative analytical results of fluorescence value of the gene editing system of Escherichia coli according to third embodiment of the present disclosure.

To verify the effect of the CRISPRi module, the dC-PLBA-EG strain and the T7-EG strain are cultured with 200 ng/ml tetracycline (Tc) to induce the performance of the CRISPRi module. In FIG. 6C, qRT-PCR analyses attest knockdown of acid production-related genes-the ptsG gene, the IdhA gene, the pfB gene and the pta gene for more than 78% in the dC-PLBA-EG strain than in the T7-EG strain. Accordingly, the dC-PLBA-EG strain exhibits significantly slower decrease of pH, faster cell growth and higher final biomass than the T7-EG strain after 48 hours of culture. In FIGS. 6D, 6E and 6F, HPLC analyses further confirm that the dC-PLBA-EG strain produced 42% lower acetate, 57% lower lactate and 100% lower formate than that of the T7-EG strain. Consequently, the results in FIGS. 6G and 6H show that the dC-PLBA-EG strain produces 367% more EGFP than the T7-EG strain after IPTG induction. These data collectively demonstrated that the gene editing system of Escherichia coli and the gene editing method of Escherichia coli of the present disclosure enable effective integration of the CRISPRi module and EGFP gene into the BL21(DE3) strain to rewire cellular pathway and enhance recombinant protein production.

### 5. Using the Gene Editing System of Escherichia coli of the Present Disclosure to Improve the Genome Engineering of MG1655 Strain to Increase Succinate Production

The MG1655 strain is a superior Escherichia coli strain for the production of bio- derived chemicals such as succinate. Since pyc gene overexpression enhances succinate production while adhE gene expression leads to byproduct (ethanol) synthesis. To understand whether the gene editing system of Escherichia coli of the present disclosure can modify the metabolic pathway in the MG1655 strain to increase the succinate production, the gene editing system of Escherichia coli of the present disclosure is used in the experiment to integrate the pyc gene into the genome and knock out the adhE gene at the same time.

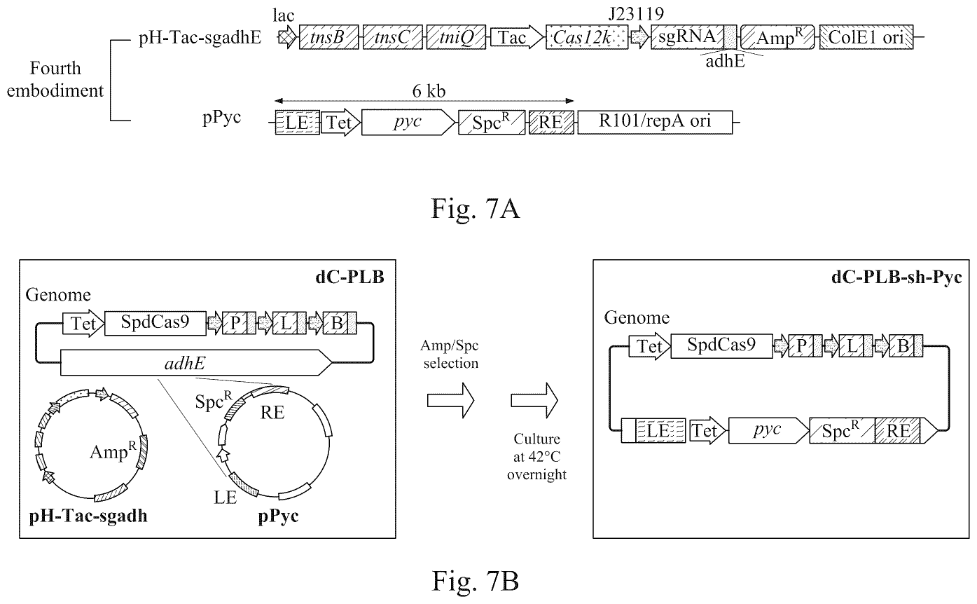

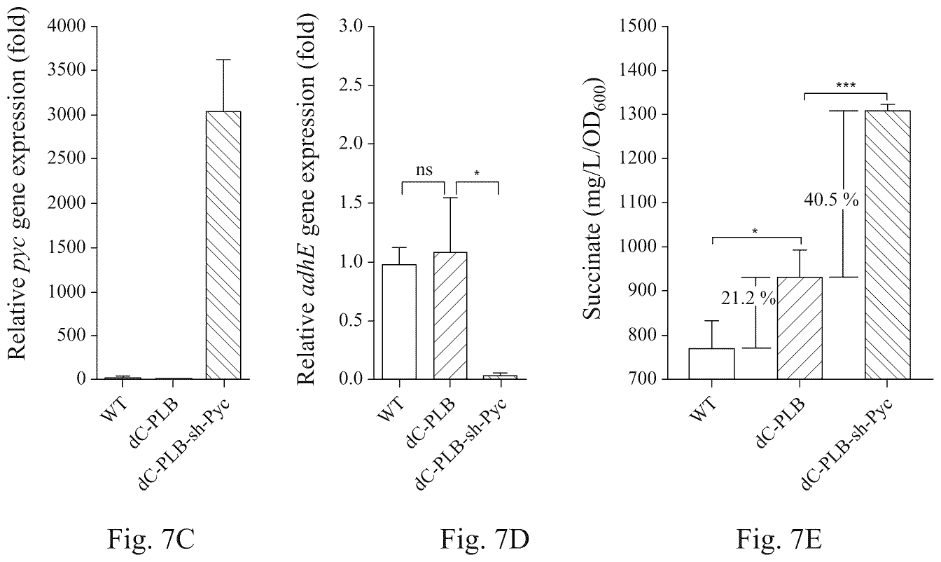

Please refer to FIG. 7A, which is a schematic view showing constructions of a helper plasmid (pH-Tac-sgadhE) and a donor plasmid (pPyc) of fourth embodiment of the present disclosure. The sequence of the spacer of the sgRNA of the pH-Tac-sgadhE is referenced as SEQ ID NO: 23, which targets the adhE gene, and the sequence of the scaffold is referenced as SEQ ID NO: 36. The pPyc includes the pyc gene (referenced as SEQ ID NO: 29) driven by the Tet promoter (referenced as SEQ ID NO: 18) and the Spc^{R} gene (referenced as SEQ ID NO: 25) composed of 6 kb expression cassette. Note that the promoter driving the Cas12k gene expression in the pH-Tac-sgadhE is changed from the T7 promoter to the Tac promoter (referenced as SEQ ID NO: 20), because the T7 promoter only worked in the BL21(DE3) strain and is not functional in other Escherichia coli strains.

Please further refer to FIG. 7B, which is an editing schematic view of a gene editing system of Escherichia coli according to fourth embodiment of the present disclosure. The pH-Tac-sgadhE and the pPyc are co-electroporated into an engineered MG1655 strain dC-PLB7 that was pre-integrated with the CRISPRi module to inhibit the ptsG gene (P), the IdhA gene (L) and the PflB gene (B). After selecting the successful gene editing colonies, the colonies are picked and cultured in 42° C. and antibiotic-free medium to remove the pH-Tac-sgadhE and the pPyc, and the resultant strain is designated as dC-PLB-sh-Pyc strain.

Please refer to FIGS. 7C to 7E. FIG. 7C shows analytical results of relative pyc gene expression of the gene editing system of Escherichia coli according to fourth embodiment of the present disclosure. FIG. 7D shows analytical results of relative adhE gene expression of the gene editing system of Escherichia coli according to fourth embodiment of the present disclosure. FIG. 7E shows analytical results of the succinate production of the gene editing system of Escherichia coli according to fourth embodiment of the present disclosure.

In FIGS. 7C to 7E, with tetracycline (Tc) induction of the CRISPRi module and the pyc gene expression, the dC-PLB-sh-Pyc strain expressed significantly higher the pyc gene expression level and barely detectable the adhE gene expression when compared with wild-type MG1655 (WT) strain and the dC-PLB strain. With Tc induction and anaerobic culture, the dC-PLB strain produces 21% more succinate than the WT strain because the ptsG gene, IdhA gene, and pflB gene are inhibited by the CRISPRi module. The dC-PLB-sh-Pyc strain modified by the gene editing system of Escherichia coli of the present disclosure produces 62% and 41% more succinate than the WT strain and the dC-PLB strain, respectively. These data indicate that the gene editing system of Escherichia coli and the gene editing method of Escherichia coli of the present disclosure enable facile concurrent the pyc gene integration and the adhE gene knockout for metabolic engineering of the MG1655 strain and succinate production.

### 6. The Gene Editing System of Escherichia coli of the Present Disclosure Enables Facile Bacmid Editing in the DH10Bac Strain and Baculovirus Engineering

Baculovirus is an insect virus but can efficiently deliver transgenic genes into target mammalian cells. Recombinant baculovirus preparation often starts from inserting the transgene into pre-designed cloning sites in the commercial bacmid (Bac-to-Bac system), which combines the features of baculovirus genome and plasmid, harbored in the DH10Bac strain. Although the commercial bacmid system is easy to use, it cannot determine the site of the integrated gene and is difficult to engineer the viral genomic backbone on the bacmid. It is difficult to use CRISPR/Cas9 to edit the bacmid backbone, presumably due to the existence of multiple bacmid copies and poor DNA repair machinery after CRISPR/Cas9-induced DNA double-strand breaks in the DH10Bac strain. To solve the problem and realize easy editing of bacmid, the gene editing system of Escherichia coli of the present disclosure is used to edit bacmid in the DH10Bac strain experimentally.

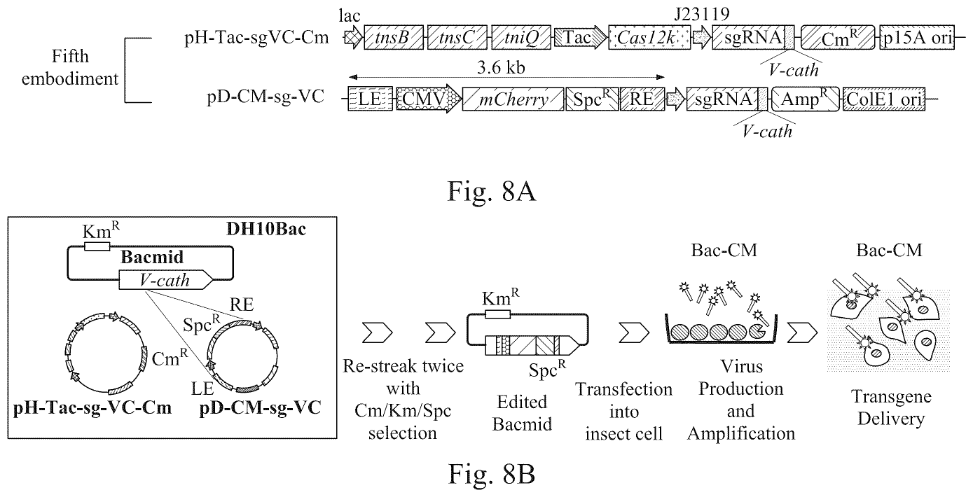

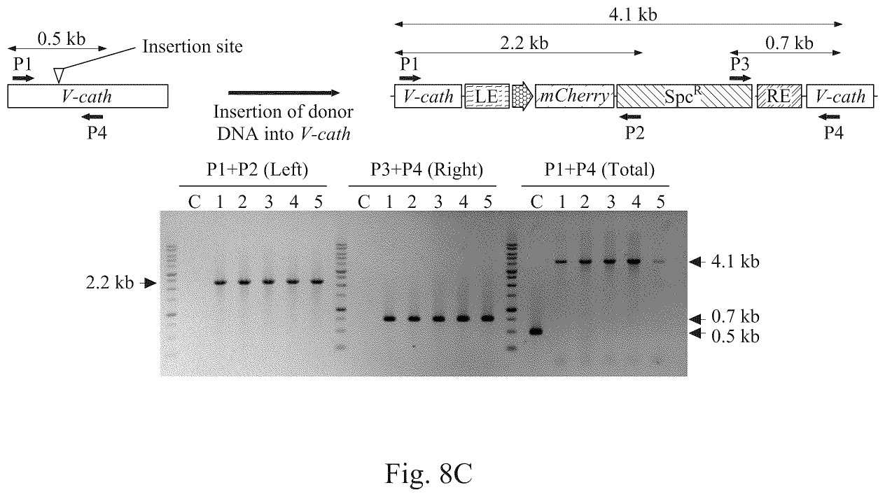

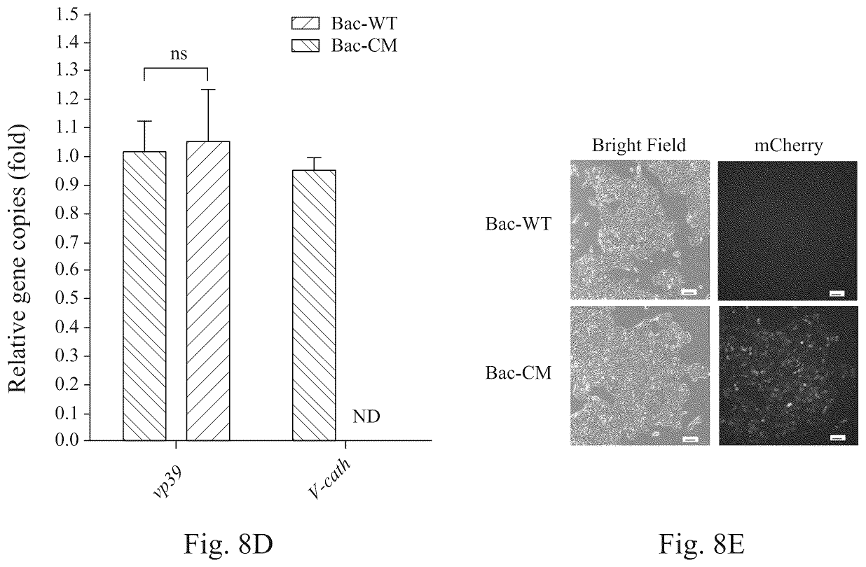

Please refer to FIG. 8A, which is a schematic view showing constructions of a helper plasmid (pH-Tac-sgVC-Cm) and a donor plasmid (pD-CM-sg-VC) of the fifth embodiment of the present disclosure. In the experiment, a foreign gene is knocked in into the bacmid-borne baculoviral V-cath gene, because it is non-essential for baculovirus replication. The pH-Tac-sgVC-Cm successively includes the transposase complex expression cassette, the Cas12k expression cassette, the first sgRNA cassette, the first antibiotic resistance gene and the first origin of replication. The transposase complex expression cassette includes the lac promoter, the tnsB gene, the tnsC gene and the tniQ gene, the Cas12k expression cassette includes the Tac promoter and the Cas12k gene, and the first sgRNA cassette includes the J23119 promoter and the sgRNA. The sequence of the spacer of the sgRNA is referenced as SEQ ID NO: 30 (targeting the V-cath gene), the sequence of the scaffold is referenced as SEQ ID NO: 36. The first antibiotic resistance gene is the chloramphenicol resistance gene (Cm^{R}) with the sequence referenced as SEQ ID NO: 31, and the first replication origin is a p15A replication origin (ori) with the sequence referenced as SEQ ID NO: 32. The pD-CM-sg-VC successively includes the left end sequence of the ShCAST transposon (referenced as SEQ ID NO: 14), the exogenous gene expression cassette, and the right end sequence of the ShCAST transposon (referenced as SEQ ID NO: 16), the second sgRNA cassette, the second antibiotic resistance gene, and the second replication origin. The exogenous gene expression cassette includes a CMV promoter with the sequence referenced as SEQ ID NO: 33, mCherry gene with the sequence referenced as SEQ ID NO: 34 and the Spc^{R} gene, and the second sgRNA cassette includes the J23119 promoter and the sgRNA with the spacer with the sequence referenced as SEQ ID NO: 30 (targeting the V-cath gene). The second antibiotic resistance gene is the AmpR gene, and the second replication origin is the ColE1 replication origin with the sequence referenced as SEQ ID NO: 13.

Please refer to FIGS. 8B and 8C, FIG. 8B is a flowchart of the baculovirus editing by a gene editing system of Escherichia coli according to fifth embodiment of the present disclosure, and FIG. 8C shows analytical results of verifying the successful integration of the gene editing system of Escherichia coli according to fifth embodiment of the present disclosure by colony PCR. In the experiment, the pH-Tac-sgVC-Cm and the pD-CM-sg-VC are co-transformed into the DH10Bac strain carrying bacmid by heat shock. Then three antibiotics, Cm/Km/Spc, are used to screen and re-streak culture, and then successful integration of the mCherry/Spc^{R} genes (total length 3.6 kb) into the V-cath gene is confirmed by colony PCR. The edited bacmid is extracted and transfected into the insect cell, Sf9 cell line, to amplify recombinant baculovirus Bac-CM (hereinafter referred to as “Bac-CM”). The colony PCR results in FIG. 8C show that the mCherry/Spc^{R} genes are indeed successfully integrated into the V-cath gene.

Please further refer to FIGS. 8D and 8E. FIG. 8D shows analytical results of the expression of the V-cath gene to be knocked out by the gene editing system of Escherichia coli according to fifth embodiment of the present disclosure. FIG. 8E shows analytical results of HEK293-FT transduced by recombinant baculovirus after being processed by the gene editing system of Escherichia coli according to fifth embodiment of the present disclosure.

The disruption of the V-cath gene in Bac-CM by comparing the copy numbers of vp39 gene, which encodes the baculovirus capsid protein and is the essential gene and the V-cath gene in the wild-type baculovirus (Bac-WT) and the Bac-CM. In FIG. 8D, the copy number of the vp39 gene is similar in the Bac-CM and the Bac-WT, that is the number of baculovirus particles is similar in the Bac-CM and the Bac-WT. However, the copy number of the V-cath gene in the Bac-CM cannot be detected, indicating that the V-cath gene has been completely disrupted. Next HEK293-FT cells transduced using Bac-WT and Bac-CM are observed by fluorescence microscope. In FIG. 8E, mCherry is expressed in most cells transduced with Bac-CM, but not in cells transduced with the Bac-WT. These data confirm that the gene editing system of Escherichia coli according to fifth embodiment of the present disclosure enables facile bacmid editing and recombinant baculovirus genetic engineering in the DH10Bac strain. The produced recombinant baculovirus can be used to efficiently introduce genes into mammalian cells.

To sum up, the gene editing system of Escherichia coli and the gene editing method of Escherichia coli of the present disclosure can be applied to various Escherichia coli strains that are important in biotechnology but difficult to edit with the CRISPR/Cas9 system, such as the BL21(DE3) strain, the W3110 strain and the W strain. The on-target efficiency of various Escherichia coli strains can be significantly improved to >90% by independently expressing the Cas12k gene using the strong promoter to increase the expression level, modifying the sequence of the replication origin, increasing the expression of the sgRNA, and reducing the gene editing temperature from 37° C. to 30° C. In addition, the gene editing system of Escherichia coli and the gene editing method of Escherichia coli of the present disclosure can integrate DNA into gene loci that are difficult to integrate using the original ShCAST system, and can integrate DNA fragments as large as 14.5 kb with 100% integration efficiency. Furthermore, the gene editing system of Escherichia coli and the gene editing method of Escherichia coli of the present disclosure are highly applicable. For example, the CRISPRi module can be integrated into the genome of the BL21(DE3) strain by using the gene editing system of Escherichia coli and the gene editing method of Escherichia coli of the present disclosure to inhibit the accumulation of acidic byproducts, thereby increasing the production of recombinant protein. The gene editing system of Escherichia coli and the gene editing method of Escherichia coli of the present disclosure can also be used to integrate the metabolic pathway gene into the genome of the MG1655 strain to increase the succinate production. In addition, the gene editing system of Escherichia coli and the gene editing method of Escherichia coli of the present disclosure can also edit the bacmid in the DH10Bac strain, so that the baculovirus backbone can be easily designed to construct a recombinant baculovirus for gene delivery to mammalian cells. Therefore, the gene editing system of Escherichia coli and the gene editing method of Escherichia coli of the present disclosure do not cause DNA double-strand breaks in Escherichia coli, and have great potential in recombinant virus engineering.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A gene editing method of Escherichia coli, comprising:
constructing a helper plasmid, wherein the helper plasmid comprises a transposase complex expression cassette, a Cas12k expression cassette, a first sgRNA cassette, a first antibiotic resistance gene and a first replication origin, the transposase complex expression cassette comprises a first promoter, a tnsB gene, a tnsC gene and a tniQ gene, the Cas12k expression cassette comprises a second promoter and a Cas12k gene, the first sgRNA cassette comprises a third promoter and a sgRNA, and the sgRNA is composed of a scaffold and a spacer;
constructing a donor plasmid, wherein the donor plasmid comprises a left end sequence of a ShCAST transposon, an exogenous gene expression cassette, a right end sequence of the ShCAST transposon, a second sgRNA cassette, a second antibiotic resistance gene and a second replication origin, the exogenous gene expression cassette comprises an exogenous gene, the second sgRNA cassette comprises a fourth promoter and the sgRNA, and the first antibiotic resistance gene and the second antibiotic resistance gene are different;
co-transforming the helper plasmid and the donor plasmid into an Escherichia coli to obtain a transformant; and
culturing the transformant for an editing time at an editing temperature, wherein the helper plasmid expresses a TnsB protein, a TnsC protein, a TniQ protein and a Cas12k protein to form a ShCAST transposon protease complex, the helper plasmid expresses the sgRNA, the donor plasmid expresses the sgRNA, and the exogenous gene is inserted into a first specific sequence of the transformant.

2. The gene editing method of Escherichia coli of claim 1, wherein the Escherichia coli is BL21(DE3) strain, BW25113 strain, MG1655 strain, W3110 strain, W strain or DH10Bac strain.

3. The gene editing method of Escherichia coli of claim 1, wherein the editing temperature is 30° C.

4. The gene editing method of Escherichia coliof claim 1, wherein the donor plasmid further comprises a CRISPRi module, and the CRISPRi module is located between the left end sequence of the ShCAST transposon and the exogenous gene expression cassette.

5. The gene editing method of Escherichia coli of claim 1, further comprising a selection step, wherein the transformant is cultured in a medium containing an antibiotic to select the transformant that is successfully co-transformed the helper plasmid and the donor plasmid.

6. The gene editing method of Escherichia coli of claim 5, wherein the antibiotic is an ampicillin (Amp), a kanamycin (Km), a spectinomycin (Spc) or a chloramphenicol (Cm).
